SEQUENCE LISTING (2 sequences)

SEQ ID NO: 1 - DNA, 163 bp, Homo sapiens
gactagaatggaaaaacggaatcctacccgatcaatgcaaattctatttctctctctccaagaggcccagatgaccagccctgacactgggagccactgggagcttcaggtgctggcccagcagtgcctggccatgttttcaagtgtacatcaggtgaccact

SEQ ID NO: 2 - DNA, 143 bp, Homo sapiens
cctactttcttgacacacagcaagaagaagtctacagagactttgtgctacttcagtgaatcttgcaacacaaagggagttcctggaccacgatggcatctggctctacgtccttgtcactcactctccctaatctgcaaaat

(19) United States
(12) Patent Application Publication
(10) Pub. No.: US 2013/0195880 A1
(43) Pub. Date: Aug. 1, 2013

(54) Identification of Polymorphisms and Their Use as Markers for Disease Susceptibility, Diagnosis and Treatment Optimization

(71) Applicants: Leonid Z. Iakoubov, San Ramon, CA (US); Henry Wilfred Lopez, Napa, CA (US); Steven Patrick Noonan, Alameda, CA (US)

(72) Inventors: Leonid Z. Iakoubov, San Ramon, CA (US); Henry Wilfred Lopez, Napa, CA (US); Steven Patrick Noonan, Alameda, CA (US)

(21) Appl. No.: 13/388,271

(22) PCT Filed: Aug. 5, 2010
(86) PCT No.: PCT/US10/02190

§ 371 Date: Mar. 12, 2013

Related U.S. Application Data
(60) Provisional application No. 61/273,512, filed on Aug. 5, 2009.

(51) Int. Cl. C12Q 1/68

(52) U.S. Cl. CPC C12Q 1/6883
USPC 424/143.1; 435/6.11; 536/24.31

(57) ABSTRACT

Provided are methods and kits to determine whether a subject is predisposed to developing a disease or condition associated with structural polymorphisms in genes CNTNAP2 and CNTNAP4, along with the methods to optimize treatment with various pharmaceutical preparations which modulate ion channels, neurite outgrowth, and myelination signaling.

### CROSS-REFERENCE TO RELATED APPLICATIONS

This application claims the benefit of U.S. Provisional application 61/273,512 filed 5 Aug. 2009 which are expressly incorporated by reference herein.

### STATEMENT REGARDING FEDERALLY SPONSORED RESEARCH OR DEVELOPMENT

Not applicable

### NAMES OF THE PARTIES TO A JOINT RESEARCH AGREEMENT

Not applicable

### SEQUENCE LISTING

The application includes two sequence listings which are included in the body of the specification as well as in the last section of this application

### BRIEF SUMMARY OF THE INVENTION

Among known common structural polymorphisms of genes CNTNAP2 and CNTNAP4 (members of neurexin superfamily), we discovered those that are substantially and significantly associated with neuro-psychiatric diseases known to have abnormal myelination as a component of their pathogenesis. The association with a disease was significant for the non-null allele variant of the polymorphism in CNTNAP2 gene (see below SEQ ID No. 1 and FIG. 1) for both gender (Table 1). The other disease association discovered, namely non-null genotype at the mentioned CNTNAP2 locus in combination with null genotype at the indicated CNTNA4 locus (see below SEQ ID No. 2 and FIG. 1 and Table 1), was strongly disease-associated in females only (Table 1).

Since these are genetic markers that could be tested starting from individual's early ontogenesis, their presence could be used to identify individuals at risk to develop the disease and to start preventive or early therapy.

### BRIEF DESCRIPTION OF THE SEVERAL VIEWS OF THE DRAWINGS

A better understanding of the present invention will be realized from the detailed description which follows, taken in conjunction with the accompanying drawings in which:

FIG. 1 shows the location of amplicons targets in the phase I study for the replication of novel risk factor candidates within known structural polymorphisms. The amplicon in FIG. 1 shows the amplicon's location at one of the distal introns of the CNTNAP2 gene, within an overlapping region of known Copy Number Variations ##36645, 22539, 39519, 43538, 6581, 7614. SEQ ID No. 1 is marked as an open circle; and

FIG. 2 shows the amplicon in one of central introns of the CNTNAP4 gene. FIG. 2 shows the location within an overlapping region of known Copy Number Variations numbers 35409, 35410, 43580, 39188. SEQ ID No. 2 is marked as an open circle.

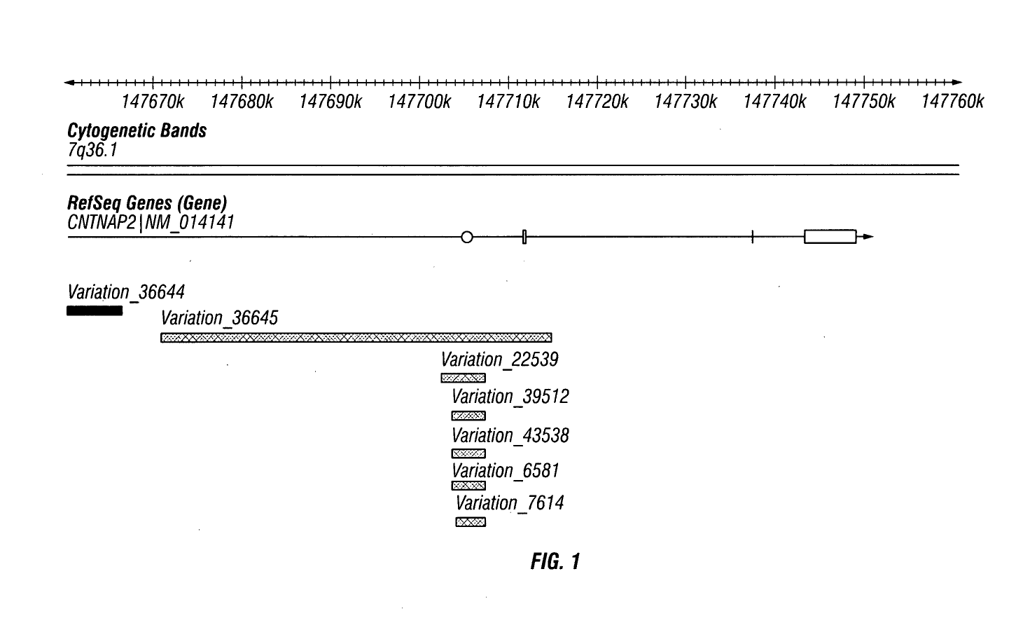

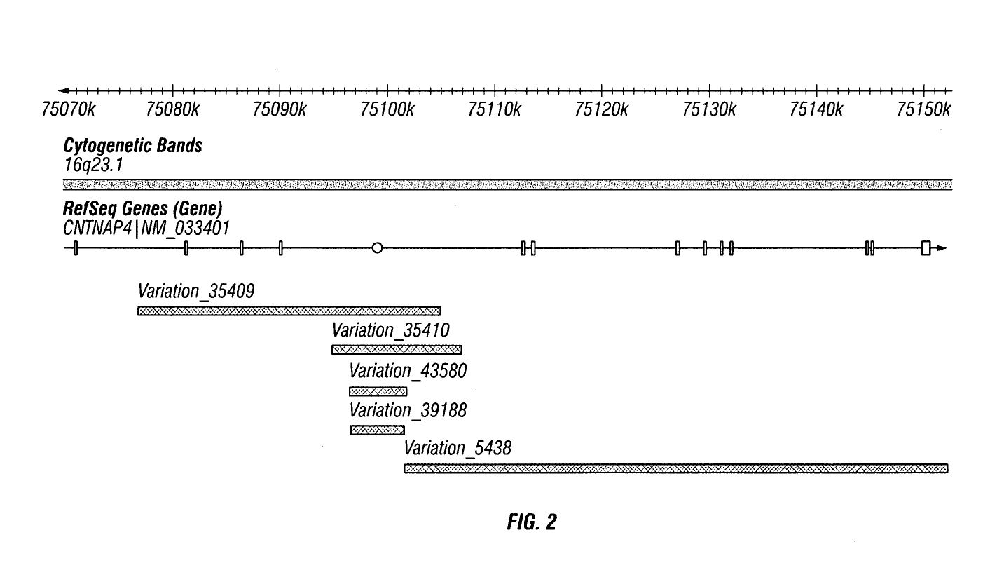

Variation 36644 in FIG. 1 is a gain. All of the other Variations in FIG. 1 and all of the Variations in FIG. 2, which are shown cross-hatched, are a loss.

### DETAILED DESCRIPTION OF THE INVENTION

### Experimental Data

We tested if any of known structural variations (copy number variations, or CNVs, deletions or insertions) from a group of selected myelination-related genes were disease-associated. Disease-related DNA samples for this testing were from patients with multiple sclerosis (MS), Schizophrenia (Sch), Bipolar disorder (BD) or Alzheimer's disease (Alz), all known to have myelination abnormalities. The DNA samples were collected from biological samples such as blood, saliva and/or buccal cells.

From 120 genes belonging to gene ontology category “myelination” (GO:0042552), 26 genes were selected that contained known structural polymorphisms (as determined with the Database of Genomic Variants (http://projects.tcag.ca/variation/). Thirty nine structural polymorphisms within the 26 selected genes were tested for disease-association using TaqMan® qPCR (Applied Biosystems, Foster City, Calif.) The PCR conditions were optimized for precise and reproducible detection of polymorphism copy number present.

The screening of samples (40 MS, 38 from patients with other above mentioned neuro-psychiatric disturbances, and 40 controls) showed that common structural polymorphisms were present in patients and controls in only ten of the 39 tested CNV-containing loci. Of those ten, two showed a trend to be disease-associated, while only two of these four (within CNTNAP2 and CNTNAP4 genes) did the trend achieve statistical significance (Table 1).

The first of these two belongs to one of CNTNAP2 gene introns at 7q36.1 location, within the overlapping region of CNVs with Ids ##22539, 36645, 39512, 43538, 6581, 7614 (numbers in accordance with the Database of Genomic Variants) (FIG. 1). The non-null genotype was statistically shown to be disease-associated, (OR=2.98; OR 95% CI=1.19-7.44; p=0.024), and the association remained independent of whether this genotype was homo- or heterozygous.

The statistical analysis of the combination of the above-mentioned structural polymorphism at gene CNTNAP2 with another structural polymorphism, at gene CNTNAP4 (an intron with 16q23.1 location; the overlapping part of CNVs with IDs ##35409, 35410, 39188, 43580; FIG. 2), resulted in the second statistically significant disease association that additionally appeared to be gender-related (Table 1). The non-null genotype at the corresponding CNTNAP2 locus in combination with null genotype at the indicated CNTNAP4 locus was strongly disease-associated in females (OR=6.65; OR 95% CI=1.35-32.61; p=0.017).

Another gender dependent association was seen in Alzheimer's patients for the non-null variant of the studied CNV in CNTNAP2 gene (Table 2). Again, only in female patients the association reached the statistical significance. Gender dependency makes this marker a potential candidate in predicting a) risks or benefits in provoking ALZ by hormone therapy used in patients for other conditions (e.g. cancer), b) individual responses to hormone therapy in ALZ patients.

The disease association in gender dependent fashion might have a prospect in predicting a) risks or benefits in provoking ALZ by hormone therapy used in patients for other conditions (e.g. cancer), and b) individual responses to hormone therapy in ALZ patients.

The association of genes from neurexin superfamily with neuro-psychiatric diseases that have myelination problems is not surprising, since at least CNTNAP2 gene is known to be myelination related. Nevertheless the data on association of particular structural polymorphisms of these genes (FIGS. 1 and 2) has never been described. The CNTNAP2 that is located in one of many gene introns has never been described. For example, the CNTNAP2 gene is one of the biggest known genes and occupies a 2.3 MB DNA segment in Chr 7. Since the two genes are also involved in ion channel regulation, neuro-psychiatric diseases are reasonably expected to be associated with the discovered polymorphisms. Epilepsy, which is such a neuro-psychiatric disease, has an imbalanced ion traffic in pathogenesis.

Though the above disease-associated polymorphisms were discovered using TaqMan qPCR technology, other PCR- or non-PCR-based technologies such as allele-specific probe hybridization, allele-specific primer extension, allele-specific amplification, sequencing, 5′ nuclease digestion, molecular beacon assay, oligonucleotide ligation assay, size analysis, single-stranded conformation polymorphism (SSCP), denaturing gradient gel electrophoresis (DGGE), and next generation sequencing could be used to identify these polymorphisms.

The polymorphisms were identified as a presence or absence of relatively small ˜200 bp segments of DNA in introns of the CNTNAP2 and CNTNAP4 genes with the following coordinates:

| CNTNAP2 (Chr7: 147705105 . . . 147705267) | |
|---|---|
| SEQ ID NO. 1 | |
| gactagaatg gaaaaacgga atcctacccg atcaatgcaa attctatttc tctctctcca | 60 |
| agaggcccag atgaccagcc ctgacactgg gagccactgg gagcttcagg tgctggccca | 120 |
| gcagtgcctg gccatgtttt caagtgtaca tcaggtgacc act | 163 |
| CNTNAP4 (Chr16: 75099169 . . . 75099311) | |
| SEQ ID NO. 2 | |
| cctactttct tgacacacag caagaagaag tctacagaga ctttgtgcta cttcagtgaa | 60 |
| tcttgcaaca caaagggagt tcctggacca cgatggcatc tggctctacg tccttgtcac | 120 |
| tcactctccc ttatctgcaa aat | 143 |

These polymorphisms could be also identified with the help of surrogate markers, like other nearby polymorphisms—SNPs or structural polymorphisms in which the linkage disequilibrium=1 or less.

The presence of the identified genetic markers in a particular patient means the presence of a corresponding marker-related disease phenotype. Hence the markers could be used to pre-select among patients for potential responders to therapies that target corresponding mechanisms like myelination, myelination signaling, ion channel function. At the same time, the absence of these markers indicate that other mechanisms in disease pathogenesis should be targeted, like immunity in MS or synaptic transmission in Schizophrenia. The method could be used in both, when selecting an existing therapy for a particular patient, and when selecting patients for testing a pharmaceutical with a specific mechanism of action in clinical trials.

The usage of the described polymorphisms in combination with known markers for a particular neuro-psychiatric disease or condition might result in better disease assessment than when a single marker is used alone.

Since both gene products are transmembrane molecules, their extracellular moieties could be potentially targeted with a monoclonal antibody or other ligands to favorably modify the function in case it is causal for the disease.

TABLE 1
Frequency (in percent) of novel disease-associated structural
polymorphisms in CNTNAP2 and CNTNAP4 genes in control
healthy population and disease (MS, Alzheimer's, bipolar
disorder, and schizophrenia. nonMS as used herein refers to
Alzheimer's, bipolar disorder and schizophrenia). In parentheses
are the number of samples with corresponding alleles out of total
samples number in the group. The Fisher exact test for 2 × 2
tables was used to calculate p values.

| | | | | Male + |
|---|---|---|---|---|
| | | Male | Female | Female |
| CNTNAP2 | MS | 47 (8/17) | 53 (10/19) | 50 (18/36) |
| non-null allele | MS + nonMS | 48 (15/31) | 52 (21/40) | 51 (36/71)¹ |
| | MS + Schiz | 55 (11/20) | 57 (12/21) | 56 (23/41) |
| | Controls | 35 (7/20) | 25 (5/20) | 30 (12/40) |
| CNTNAP4 | MS | 41 (10/17) | 21 (15/19) | 31 (25/36) |
| null allele | MS + nonMS | 48 (16/31) | 30 (28/40) | 38 (44/71) |
| | MS + Schiz | 45 (12/20) | 19 (17/21) | 31 (29/41) |
| | Controls | 35 (13/20) | 40 (12/20) | 38 (25/40) |
| Combination: | MS | 18 (3/17) | 53 (10/19) | 36 (13/36) |
| CNTNAP2 | MS + nonMS | 16 (5/31) | 42 (17/40)² | 31 (22/71) |
| non-null + | MS + Schiz | 25 (5/20) | 57 (12/21) | 39 (17/41) |
| CNTNAP4 | Controls | 30 (6/20) | 10 (2/20) | 20 (8/40) |
| null | | | | |

¹OR = 2.40; OR 95% CI = 1.05-5.45; p = 0.046;
²OR = 6.65; OR 95% CI = 1.35-32.61; p = 0.017;

TABLE 2
The association of the CNV in CNTNAP2 gene with Late Onset
Alzheimer's Disease (LOAD). Data were obtained in a Confirmation
study on 72 LOAD and 90 matched normal control samples, both
sample sets from National Cell Repository for AD (NCRAD).

| | OR | 95% CI | p-value |
|---|---|---|---|
| Males + Females | 1.46 | 0.82-2.56 | 0.196 |
| Males | 1.24 | 0.53-2.87 | 0.674 |
| Females | 2.44 | 1.11-5.33 | 0.029 |

What is claimed is:

1. A method of determining whether a human has an increased risk for developing a degenerative neurological or psychiatric condition comprising testing nucleic acid from said human for the presence or absence of a CNV polymorphism in the genes CNTNAP2 and CNTNAP4 of said human's nucleic acid, by detecting the absence of either SEQ ID No. 1 or SEQ ID NO. 2, and correlating the presence or absence of said polymorphism with said human having an increased risk for developing a degenerative neurological or psychiatric condition.

2. The method of claim 1 wherein said biological sample is blood, saliva, or buccal cells.

3. The method of claim 1 wherein said testing step comprises nucleic acid amplification.

4. The method of claim 3 wherein said nucleic acid amplification is carried out by polymerase chain reaction.

5. The method of claim 1 wherein said correlating step is performed by a computer.

6. The method of claim 1, wherein said testing is carried out by a process selected from the group consisting of allele-specific probe hybridization, allele-specific primer extension, allele-specific amplification, sequencing, 5′ nuclease digestion, molecular beacon assay, oligonucleotide ligation assay, size analysis, single-stranded conformation polymorphism (SSCP), denaturing gradient gel electrophoresis (DGGE), and next generation sequencing.

7. A method of determining whether a human has an increased risk for developing a degenerative neurological or psychiatric condition, comprising testing nucleic acid from said human for the presence or absence of copy number variation (CNV) at pos 1 or 2 or any other polymorphism in linkage disequilibrium with a copy number polymorphism at positions No 1 or 2, and correlating the presence or absence of said CNV polymorphism with said human having an increased risk for developing said degenerative neurological or psychiatric condition.

8. An isolated polynucleotide which specifically hybridizes to a segment of a nucleic acid molecule, wherein said segment belongs to polymorphic CNVs as shown in FIG. 1 or 2.

9. A kit for detecting a polymorphism in a nucleic acid, comprising the polynucleotide of claim 8, a buffer, and an enzyme.

10. A method of claim 1 wherein said human is female.

11. A method of claim 1 wherein said human is male.

12. A method of claim 1 wherein said degenerative neurological or psychiatric condition is selected from the group consisting of Alzheimer's disease, schizophrenia, bipolar disorder and multiple sclerosis.

13. The method of claim 12 wherein the condition is Alzheimer's disease.

14. The method of claim 12 wherein the condition is bipolar disorder.

15. The method of claim 12 wherein the condition is schizophrenia.

16. The method of claim 12 where in the condition is multiple sclerosis.

17. A method for administering a therapeutic agent, the method comprising the genotyping of CNV polymorphisms in position 1 and 2 of genes CNTNAP2 and CNTNAP4, correspondingly, in the nucleic acid of at least one individual, and either administering the therapeutic agent to the individual if the results of genotyping indicated that said individual is not likely to respond to said therapeutic agent.

18. A method for conducting a clinical trial in which a therapeutic agent is evaluated comprising genotyping one or more polymorphisms at position 1 and 2 of genes CNTNAP2 and CNTNAP4 in the nucleic acid of one or more individuals, and including in the clinical trial those individuals for whom the results of said genotyping indicated are likely to respond to said therapeutic agent and/or excluding from the clinical trial those individuals for whom the results of said genotyping indicated are not likely to respond to said therapeutic agent.
